(19) United States
(12) Plant Patent Application Publication
(10) Pub. No.: US 2002/0092082 P1
(43) Pub. Date: Jul. 11, 2002

(54) Petunia plan named 'Dancasye'

(75) Inventor: Gabriel Danziger, Moshav Nir-Zvi (IL)

Correspondence Address: FOLEY AND LARDNER, SUITE 500, 3000 K STREET NW, WASHINGTON, DC, 20007, US

(73) Assignee: Danziger - "Dan" Flower Farm

(21) Appl. No.: 10/075,609

(22) Filed: Feb. 15, 2002

Related U.S. Application Data
(63) Continuation of application No. 09/730,779, filed on Dec. 7, 2000, now abandoned.

(51) Int. Cl. A01H 5/00

(52) U.S. Cl. PLT/356

(57) ABSTRACT

The new petunia ‘Dancasye’ is characterized by white flowers with a yellow eye; a spreading but slightly upright growth habit and a plant width at maturity of about two feet.

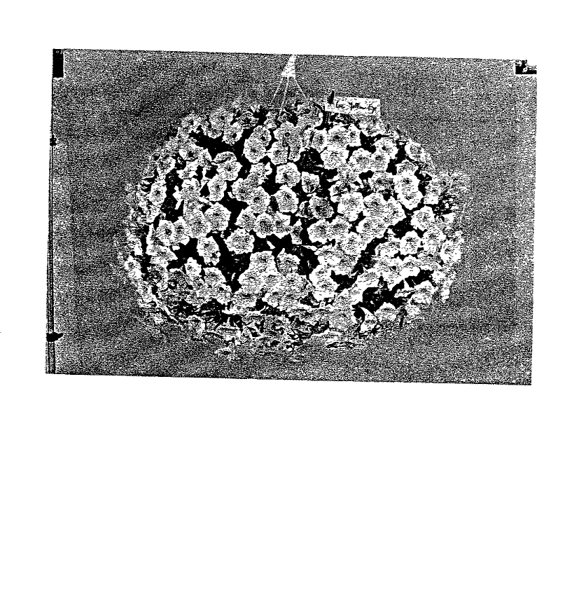

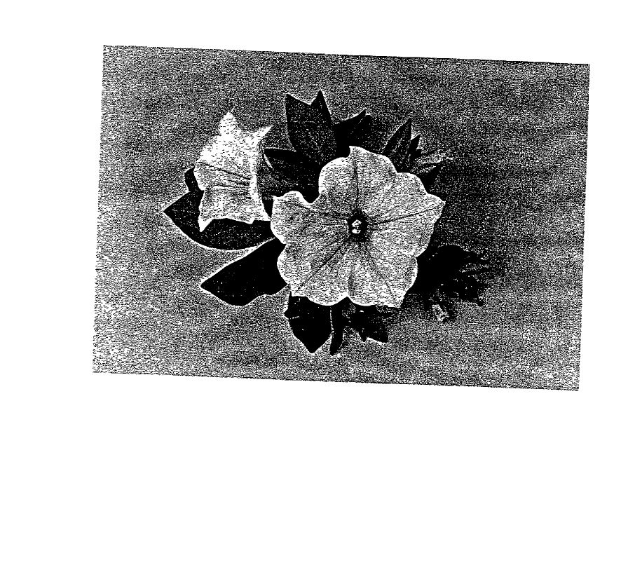

### BACKGROUND OF THE INVENTION

The present invention comprises a new and distinct cultivar of Petunia, known by the cultivar name ‘Dancasye’.

The new cultivar ‘Dancasye’ was originated from a hybridization made by the inventor in a controlled breeding program in Mishmar Hashiva, Israel in 1996. The female parent was proprietary Hybrid selection No. 13. The male parent of ‘Dancasye’ was proprietary Hybrid selection No. 822. ‘Dancasye’ was discovered and selected as one flowering plant within the progeny of the stated cross by or under the supervision of the inventor, Gabriel Danziger on May 1996 in a controlled environment in Mishmar Hashiva, Israel.

The first act of asexual reproduction of ‘Dancasye’ was accomplished when vegetative cuttings were taken from the initial selection on September 1996 in a controlled environment in Mishmar Hashiva, Israel by a technician working under formulations established and supervised by the inventor. Horticultural examination of selected units initiated February 1997 has demonstrated that the combination of characteristics as herein disclosed for the ‘Dancasye’ are firmly fixed and are retained through successive generations of asexual reproduction.

‘Dancasye’ has not been observed under all possible environmental conditions. The phenotype may vary somewhat with variations in environment such as temperature, light intensity and day length, however, without any variations in genotype. The following observations, measurements and comparisons describe the plants grown in Mishmar Hashiva, Israel, under conditions which approximate those generally used in commercial practice.

### BRIEF DESCRIPTION OF THE INVENTION

The following traits have been repeatedly observed and are determined to be basic characteristics of ‘Dancasye’ which in combination distinguish this petunia as a new and distinct cultivar:

1. flower color—white with a yellow eye;

2. flower type—single, medium;

3. flowering time—early, all year round;

4. growth habit—spreading with a slight upright tendancy, well-branched, vigorous plant;

5. good weather tolerance;

6. in its maturity, plant reaches a height of approximately 2 feet across; and

7. suitable for hanging baskets and for ground covering.

Of the many commercial cultivars known to the present inventor(s), the most similar in comparison to ‘Dancasye’ is ‘Prism Sunshine’. Reference is made to attached Chart A which compares certain characteristics of ‘Dancasye’ to those same characteristics of ‘Prism Sunshine’. In comparison to ‘Prism Sunshine’, ‘Dancasye’ has different corolla coloration and a more cascading growth habit.

### BRIEF DESCRIPTION OF THE DRAWING

The accompanying photographic drawing(s) show typical flower and foliage characteristics of ‘Dancasye’, with colors being as true as possible with illustrations of this type. Sheet 1 is a side view of the plant. Sheet 2 is a close-up of a flower from the plant.

### DETAILED BOTANICAL DESCRIPTION

In the following description color references are made to The Royal Horticultural Society Color Chart (RHS). The color values were determined between 10:00 and 12:00 a.m. on Aug. 9, 2000 under full sunlight at Mishmar Hashiva, Israel.

Origin: Mishmar Hashiva, Israel

Classification:

Botanical.—A hybrid of petunia—species unknown.

Commercial.—Petunia cultivar ‘Petunia Hybrida’.

Plant:

Growth Habit.—spreading with a slight upright tendancy.

Plant Height.—6-8 inches.

Spreading Area.—30 inches.

Blooming period.—All year round in moderate climate.

Stem:

Thickness.—4 mm.

Color.—yellow-green RHS 146A.

Pubescence.—yes.

Branching.—freely.

Length of internode.—10-15 mm.

Leaf:

Arrangement on stem.—alternate.

Shape.—Eliptic.

Size.—Length: 75 mm; Width: 45 mm.

Thickness.—0.2 mm.

Color.—yellow-green RHS 147A.

Markings.—none.

Pubescence.—yes.

Flower:

Orientation at opening.—upright.

Type.—single.

Shape.—regular rounded, corolla is united with 5 lobes and a long straight tube.

Diameter.—65 mm.

Color.—Upper: RHS 155B Bottom of inside surface of throat: yellow RHS 9A.

Peduncle.—15 mm; yellow-green RHS 146A.

Reproductive organs:

Pistil.—1, yellow-green RHS 145C.

Stamens.—5.

Anther.—yellow RHS 11 C.

Filament.—yellow-green RHS 145D.

Resistance: It is a new cultivar highly resistant to: rain and heat as well as disease under regular growing conditions

I claim:

1. A new and distinct Petunia plant named ‘Dancasye’, substantially as illustrated and described herein.
